(19) United States
(12) Patent Application Publication
(10) Pub. No.: US 2021/0366266 A1
(43) Pub. Date: Nov. 25, 2021

(54) System and Method for Providing Real-Time Communication with Emergency Services

(71) Applicants: Mack Owens, Hampton, VA (US); Dorothy Owens, Hampton, VA (US)

(72) Inventors: Mack Owens, Hampton, VA (US); Dorothy Owens, Hampton, VA (US)

(21) Appl. No.: 17/397,852

(22) Filed: Aug. 9, 2021

Related U.S. Application Data
(63) Continuation-in-part of application No. 16/716,483, filed on Dec. 16, 2019.
(60) Provisional application No. 62/798,939, filed on Jan. 30, 2019.

(51) Int. Cl. G08B 25/00; H04W 4/20; H04W 12/06; G08B 27/00; H04W 4/90

(52) U.S. Cl. CPC G08B 25/006; H04W 4/20; H04W 4/90; G08B 27/001; H04W 12/06

(57) ABSTRACT

A system and method for providing real-time communication with emergency services allows users to open a line of communication with appropriate authorities during an emergency situation. A user has the ability to broadcast their location and video of their situation to authorities and social media, thus improving communication during police encounters and medical emergencies. An emergency response system connects users and emergency responders through an emergency. Further, the emergency response system allows users to send and receive a plurality of media content to/from emergency responders. In the preferred embodiment, a software application, preferably referred to as All Eyes on You, is provided, which allows users to store, share, and/or receive a plurality of data which can be transmitted through various media formats. The emergency response system of the method further enables the communication between a plurality of third-party systems involved in an emergency.

The current application is a continuation-in-part (CIP) application of a U.S. non-provisional application Ser. No. 16/716,483 filed on Dec. 16, 2019. The U.S. non-provisional application Ser. No. 16/716,483 claims a priority to a U.S. provisional application Ser. No. 62/798,939 filed on Jan. 30, 2019.

### FIELD OF THE INVENTION

The present invention generally relates to systems and methods for emergency response and communications. More specifically, the present invention relates to systems and methods for the sharing of media between users and emergency responders during various emergency situations.

### BACKGROUND OF THE INVENTION

Emergency response communication can be a critical aspect of proper emergency management. Quick and efficient emergency response communication can often save lives or prevent greater casualties. Various emergency response systems have been provided over the years which enable the transmission of emergency-related information between different parties involved. Traditional emergency response systems allow for users to contact emergency responders such as police or the fire department when an emergency occurs. Newer emergency response systems have been provided which increase the efficiency and timing of emergency response communication by automating various processes of the emergency response.

Unfortunately, most emergency response systems fail to set up proper and efficient communication between the emergency responders and the users throughout the emergency. It is common for the communication between users and emergency responders to be limited, with some initial communication when users request emergency response and some communication after emergency response has been deployed. This leaves users often in the dark until the emergency responders come to their aid. On the other hand, emergency responders must rely with the initial information provided to them which can often be incomplete or even incorrect. Incomplete or incorrect information can limit the effectiveness of the emergency response, which can in turn increase the severity of the emergency.

An objective of the present invention is to provide a system and method for the sharing of media between users and emergency responders during various emergency situations. The present invention provides an emergency response system which connects users and emergency responders through an emergency. Further, the emergency response system allows users to send and receive a plurality of media content to/from emergency responders. In the preferred embodiment of the present invention, the present invention provides a software application, preferably referred to as All Eyes on You, which allows users to store, share, and/or receive a plurality of data which can be transmitted through various media formats. The emergency response system of the present invention further enables the communication between a plurality of third-party systems involved in an emergency.

### BRIEF DESCRIPTION OF THE DRAWINGS

FIG. 1 is a block diagram illustrating the system of the present invention.

FIG. 2 is a flowchart illustrating the overall process for the method of the present invention.

FIG. 3 is a continuation of FIG. 2.

FIG. 4 is a flowchart illustrating the subprocess of handling a theft case.

FIG. 5 is a flowchart illustrating the subprocess of handling a medical emergency case.

FIG. 6 is a flowchart illustrating the subprocess of an individual handling a police encounter.

FIG. 7 is a flowchart illustrating the subprocess of displaying relevant information to the individual during a police encounter.

FIG. 8 is a flowchart illustrating the subprocess of displaying relevant information to the individual.

FIG. 9 is a flowchart illustrating the subprocess of appending images and video files to an emergency event.

FIG. 10 is a flowchart illustrating the subprocess of providing live-view footage to a responder during an emergency event.

FIG. 11 is a flowchart illustrating the subprocess of providing footage from external cameras.

FIG. 12 is a flowchart illustrating the subprocess of broadcasting live-view data to many users.

FIG. 13 is a flowchart illustrating the subprocess of providing a connection operator to facilitate connecting responders to individuals.

FIG. 14 is a flowchart illustrating the subprocess of providing a connection operator to facilitate connecting individuals to responders.

FIG. 15 is a flowchart illustrating the subprocess of displaying a graphical interface to an operator.

FIG. 16 is a flowchart illustrating the subprocess of outputting emergency event information publicly.

FIG. 17 is a flowchart illustrating the subprocess of providing a connection operator to facilitate connecting individuals to personal accounts.

FIG. 18 is a flowchart illustrating the subprocess of displaying a graphical interface to an operator.

FIG. 19 is a flowchart illustrating the subprocess of outputting emergency event information publicly.

FIG. 20 is a flowchart illustrating the subprocess of enabling voice activation of different functions of the present invention.

FIG. 21 is a flowchart illustrating the subprocess of enabling three-way live communications.

FIG. 22 is a flowchart illustrating the subprocess of applying a filter to information sent during an emergency event.

### DETAILED DESCRIPTION OF THE INVENTION

All illustrations of the drawings are for the purpose of describing selected versions of the present invention and are not intended to limit the scope of the present invention.

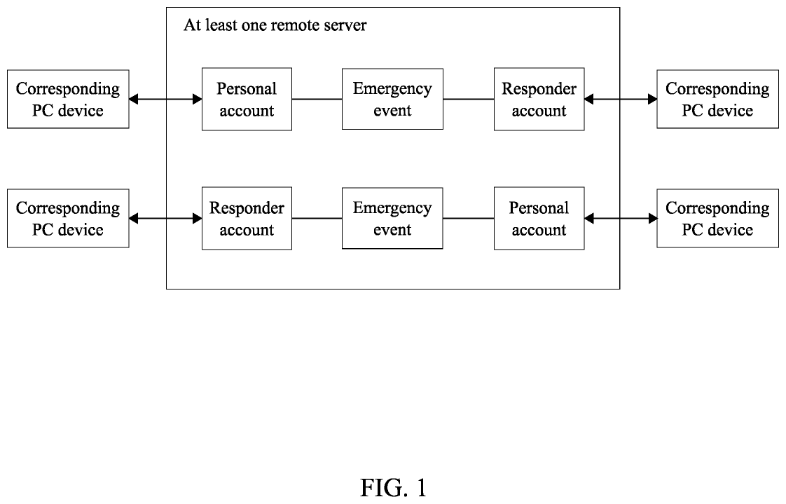

The present invention is a system and method for providing real-time communication with emergency services that allows a user to open a line of communication with appropriate authorities during an emergency situation. A user has the ability to broadcast their location and video of their situation to authorities and social media, thus improving communication during police encounters and medical emergencies, as represented in FIG. 1. The system of the present invention includes a plurality of user accounts managed by at least one remote server, wherein each user account includes authenticated information and is associated with a corresponding personal computing (PC) device, and wherein the plurality of user accounts includes a plurality of personal accounts and a plurality of responder accounts (Step A). The plurality of user accounts is the set of general information, including, but not limited to, names, ages, demographic data, and more, as well as any necessary credentials associated with each user. The at least one remote server relates to a set of wireless computing devices capable of collecting, storing, applying logic, and relaying data between wirelessly connected devices. The authenticated information is any or all of demographical information, medical history, professional history, legal history, age, weight, height, birthday, emergency contact information, selected personal images, or a variety of other personal information not listed or suggested in the above categories that can prove valuable in the event of an emergency. The corresponding PC device is any computer, laptop, smartphone, or other such internet-enabled device that allows the user to communicate to the at least one remote server. The plurality of personal accounts is a set of user accounts for anyone that may potentially experience an emergency situation. Conversely, the plurality of responder accounts is another set of user accounts for anyone that may respond to an emergency situation such as police officers, emergency medical technicians, doctors, firefighters, or a variety of other professionals in the field of emergency services. This arrangement enables each user to interact with the at least one remote server using the corresponding PC device.

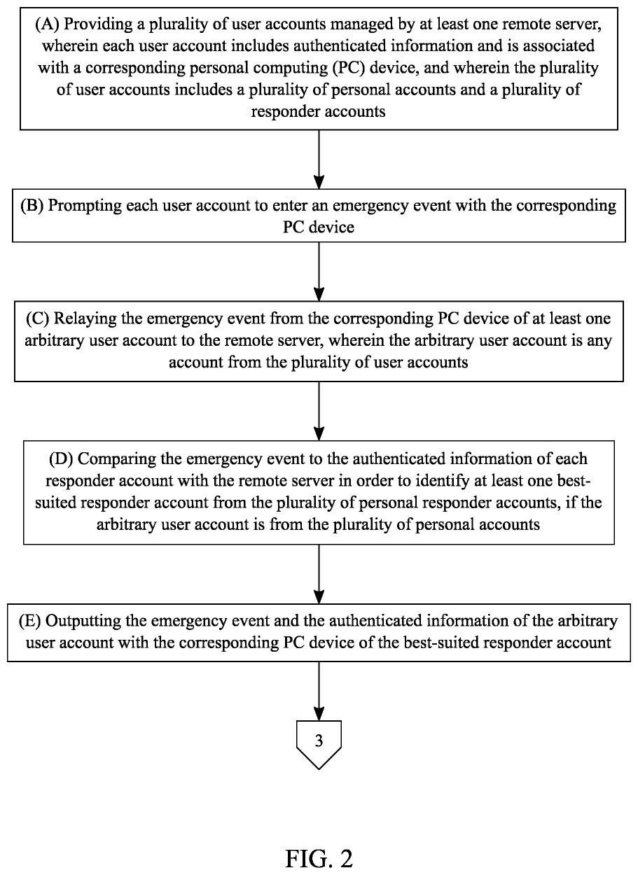

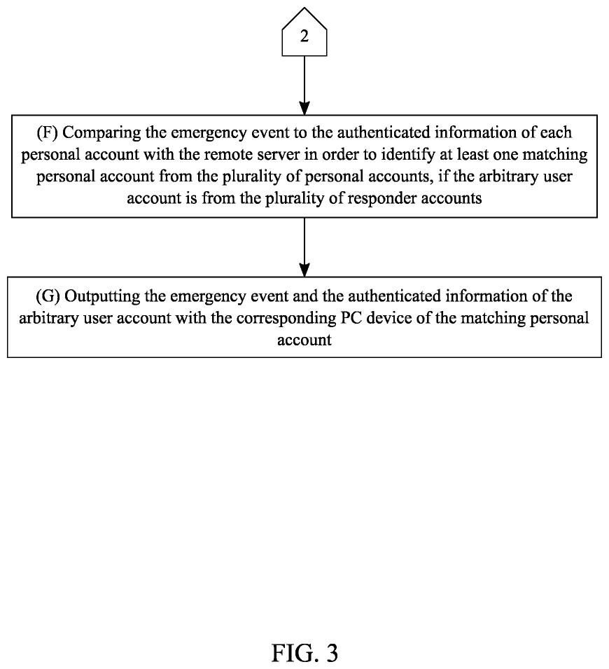

As can be seen in FIGS. 2 and 3, the method of the present invention follows an overall process in order to allow the plurality of personal accounts to efficiently and effectively communicate with the plurality of responder accounts. Each user account is prompted to enter an emergency event with the corresponding PC device (Step B). Thus, a user may be provided access to the necessary information, either by entering details or sending previously stored information, to begin to open a line of communication with relevant parties. The emergency event is relayed from the corresponding PC device of at least one arbitrary user account to the remote server, wherein the arbitrary user account is any account from the plurality of user accounts (Step C). This arrangement enables the remote server to handle data received from the arbitrary user account. Next, the emergency event is compared to the authenticated information of each responder account with the remote server in order to identify at least one best-suited responder account from the plurality of responder accounts, if the arbitrary user account is from the plurality of personal accounts (Step D). Thus, the present invention distinguishes between the plurality of personal accounts and the plurality of responder accounts and identifies the appropriate party or parties to contact. The emergency event and the authenticated information of the arbitrary user account is outputted with the corresponding PC device of the best-suited responder account (Step E). The best-suited responder account therefore responds to an emergency situation with the information provided from the arbitrary user account. Alternatively, the emergency event is compared to the authenticated information of each personal account with the remote server in order to identify at least one matching personal account from the plurality of personal accounts, if the arbitrary user account is from the plurality of responder accounts (Step F). Thus, the present invention allows a given responder account, particularly an account belonging to a police officer, to identify the appropriate party or parties involved in an incident. Such an arrangement is particularly advantageous in the event that a responder account belongs to a police officer who wants to investigate a pulled-over vehicle or similar incident. The emergency event and the authenticated information of the arbitrary user account are outputted with the corresponding PC device of the matching personal account (Step G). Thus, the responder account is provided with the information necessary to safely approach a situation, knowing the person in the vehicle as well as the person's medical and criminal history before approaching.

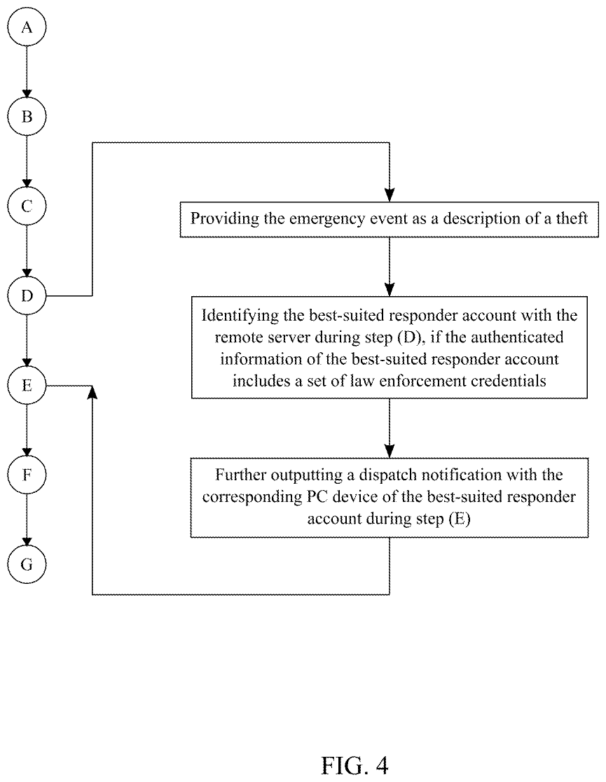

In an embodiment of the present invention, the emergency event involves criminal activity. In this embodiment, the emergency event is provided as a description of a theft. The description of theft may be details of any incident involving burglary, robbery, or any similar activity involving unjust claiming of objects by various parties. The best-suited responder account is identified with the remote server during Step D, if the authenticated information of the best-suited responder account includes a set of law enforcement credentials, as represented in FIG. 4. The set of law-enforcement credentials indicates that the best-suited responder account belongs to the appropriate authorities (e.g. local police, state police, federal agents, etc.) that are able to perform the proper law-enforcement services for a theft. This step may include a filter to differentiate between a plurality of qualified responder accounts, sorting through the plurality of qualified responder accounts by various criteria including, but not limited to, distance, arrest records, disciplinary actions, and years of experience, each criterium preferably weighted according to the settings of each personal account. A dispatch notification is further outputted with the corresponding PC device of the best-suited responder account during Step E. Thus, the best-suited responder account is notified to immediately dispatch and respond to an emergency event by the corresponding PC device of the at least one arbitrary user account, as well as other responder accounts made aware of the emergency event.

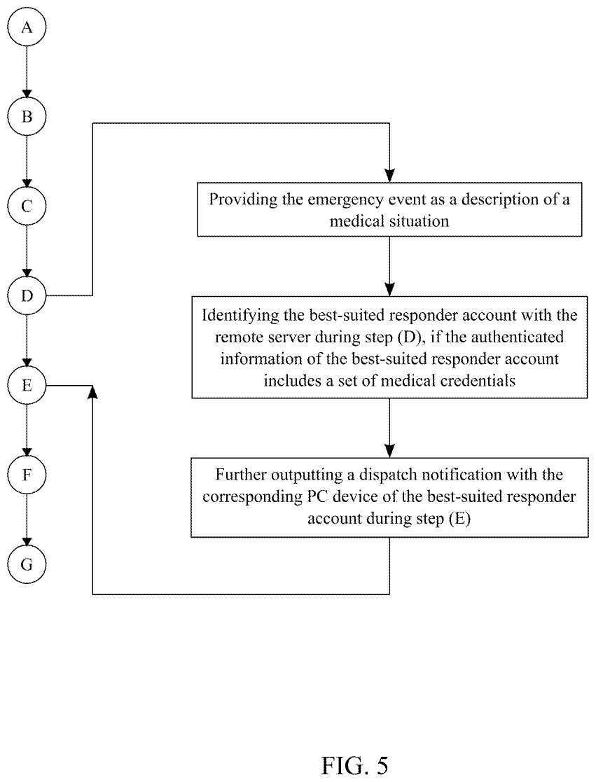

In another embodiment of the present invention, the emergency event relates to a medical emergency. The emergency event is provided as a description of a medical situation. A medical situation may relate to events occurring to the operator of the personal account directly or may relate to individuals near to the operator of the personal account. The best-suited responder account is identified with the remote server during Step D, if the authenticated information of the best-suited responder account includes a set of medical credentials, as represented in FIG. 5. The set of medical credentials indicates that the best-suited responder account belongs to the appropriate authorities (e.g. paramedics, nurses, doctors, etc.) that are able to administer the proper medical care for a medical emergency. This step may include a filter to differentiate between a plurality of qualified responder accounts, sorting through the plurality of qualified responder accounts by various criteria including, but not limited to, distance, specialization, and years of experience, each criterium preferably weighted according to the settings of each personal account. A dispatch notification is further outputted with the corresponding PC device of the best-suited responder account during Step E. Thus, the best-suited responder account is notified to immediately dispatch and respond to an emergency event by the corresponding PC device of the at least one arbitrary user account, as well as other responder accounts made aware of the emergency event.

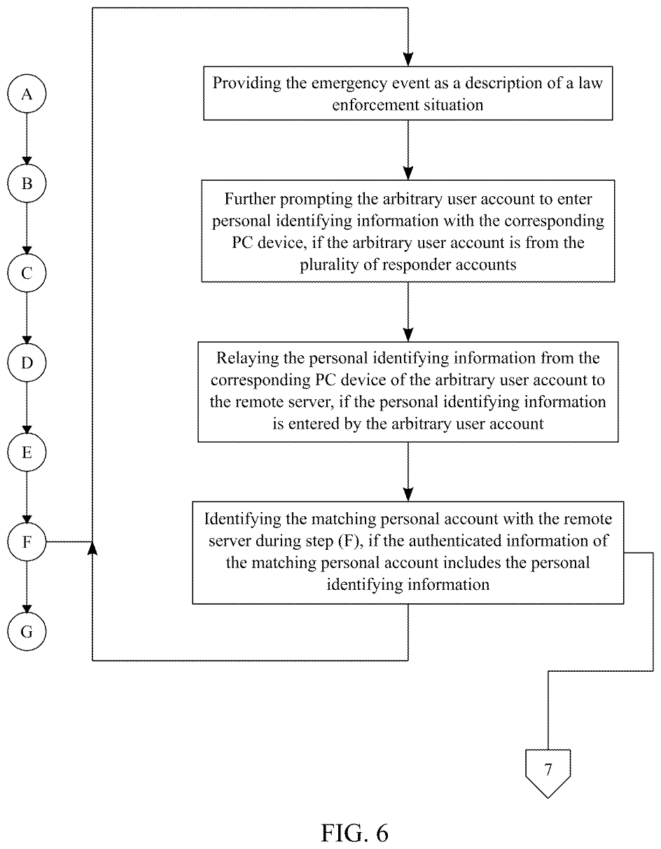

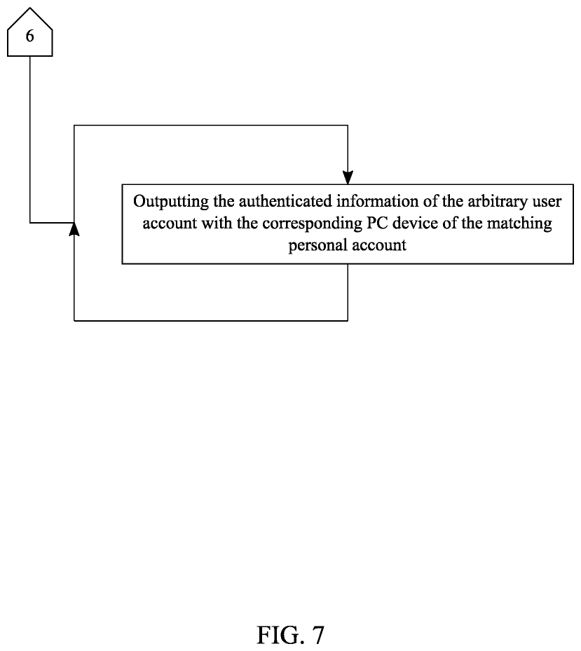

Another embodiment addresses the issue of a police officer or other responder account coming into contact with an unknown party in the line of work. In order to address this circumstance, the emergency event is provided as a description of a law enforcement situation, as represented in FIG. 6. A law enforcement situation can include traffic incidents, hostile incidents, hold-ups, or a variety of other incidents in which a police officer must approach an unknown entity. The arbitrary user account is prompted to enter personal identifying information with the corresponding PC device, if the arbitrary user account is from the plurality of responder accounts. Personal identifying information may include, but is not limited to, name, age, address, phone number, license number, and more. In this way, the necessary and relevant information that may help the police officer is made available directly from the arbitrary user account. The personal identifying information is relayed from the corresponding PC device of the arbitrary user account to the remote server, if the personal identifying information is entered by the arbitrary user account. This arrangement allows for storage of metadata regarding the personal identifying information, potentially including time of submission, geolocation, internet service provider, internet protocol (IP) address, and more. Finally, the matching personal account is identified with the remote server during Step F, if the authenticated information of the matching personal account includes the personal identifying information. In this way, the relevant information is collected and prepared for delivery to the police officer or other law enforcement official before any direct interaction between the police officer and the arbitrary user account occurs. In an exemplary embodiment of this situation, the authenticated information of the arbitrary user account is outputted with the corresponding PC device of the matching personal account, as represented in FIG. 7. This arrangement ensures that the appropriate party is aware of the success of the information sharing in relation to the emergency event.

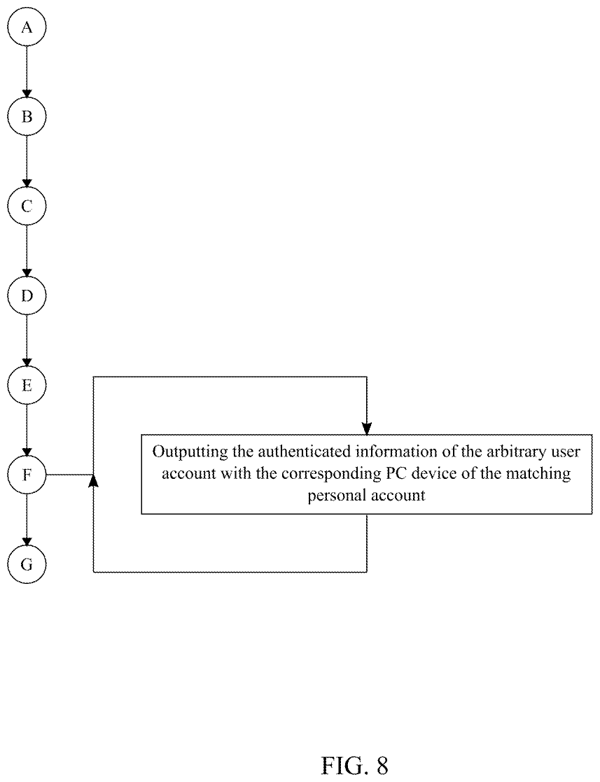

In many situations, it is advantageous to keep the sender informed about the successful transfer of an emergency event, along with any situation-specific information. To this end, the authenticated information of the arbitrary user account is outputted with the corresponding PC device of the matching personal account, as represented in FIG. 8. This arrangement ensures that the appropriate party is aware of the success of the information sharing in relation to the emergency event.

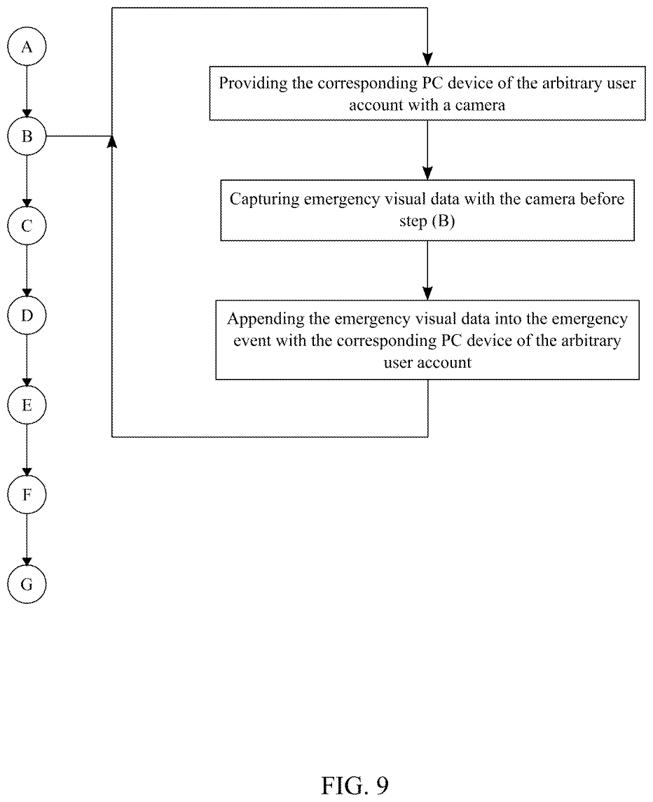

The judicious application of information gathering technologies has become an integral component of data collection, and the communication of images and video can drastically improve the ability of first responders to quickly address a situation. To improve the potential for collection of relevant data, the corresponding PC device of the arbitrary user account is provided with a camera, as represented in FIG. 9. The camera may be any image-capturing technology, including static image collection and video collection. The camera may further be integrated into existing tools and devices, such as mirrors or other objects commonly utilized in stealthier video recording. Emergency visual data is captured with the camera before Step B. The emergency visual data may include, but is not limited to, data that captures a video recording in order to depict what is going on during a medical emergency. Thus, each user account is capable of visually documenting transpiring proximal events. The emergency visual data is then appended into the emergency event with the corresponding PC device of the arbitrary user account. In this way, the emergency visual data is provided for subsequent usage by appropriate user accounts.

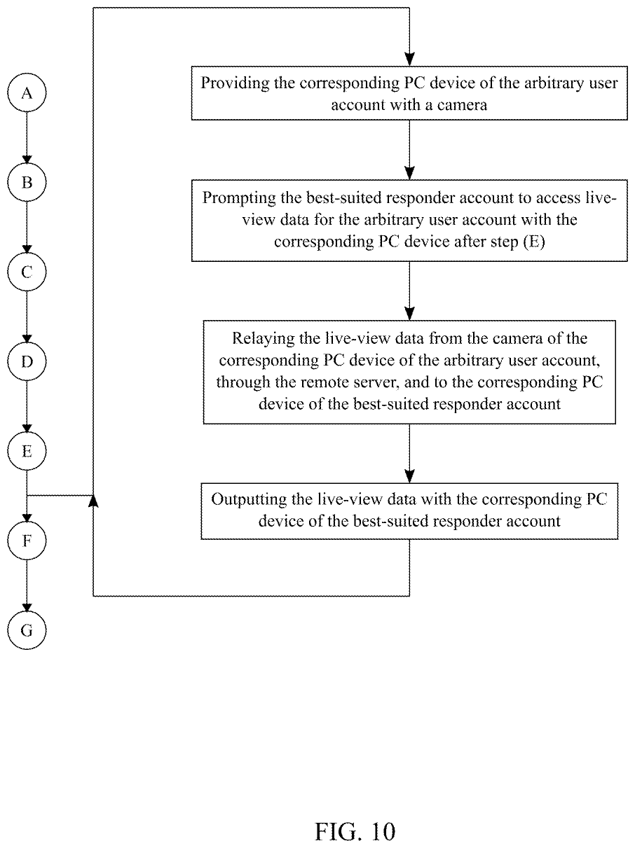

Live data, particularly as it relates to video footage, is invaluable as a primary source for communicating necessary information. To incorporate live data communication capabilities, the corresponding PC device of the arbitrary user account is provided with a camera, as represented in FIG. 10. The camera is any of a variety of video recording devices, optionally capturing audio data as well through an attached microphone or sound sensor. The camera may further be integrated into existing tools and devices, such as mirrors or other objects commonly utilized in stealthier video recording. The best-suited responder account is prompted to access live-view data for the arbitrary user account with the corresponding PC device after Step E. The live-view data is a set of information streamed from the camera. The live-view data is relayed from the camera of the corresponding PC device of the arbitrary user account, through the remote server, and to the corresponding PC device of the best-suited responder account. Thus, the live-view data is communicated through the remote server, allowing for creation and storage of data including times, locations, and more. Finally, the live-view data is outputted with the corresponding PC device of the best-suited responder account. This way, the best-suited responder account can watch events unfold in real-time. Such a capability is advantageous in many situations, such as during medical emergencies, when every second counts and a real-time instructive video could save a life.

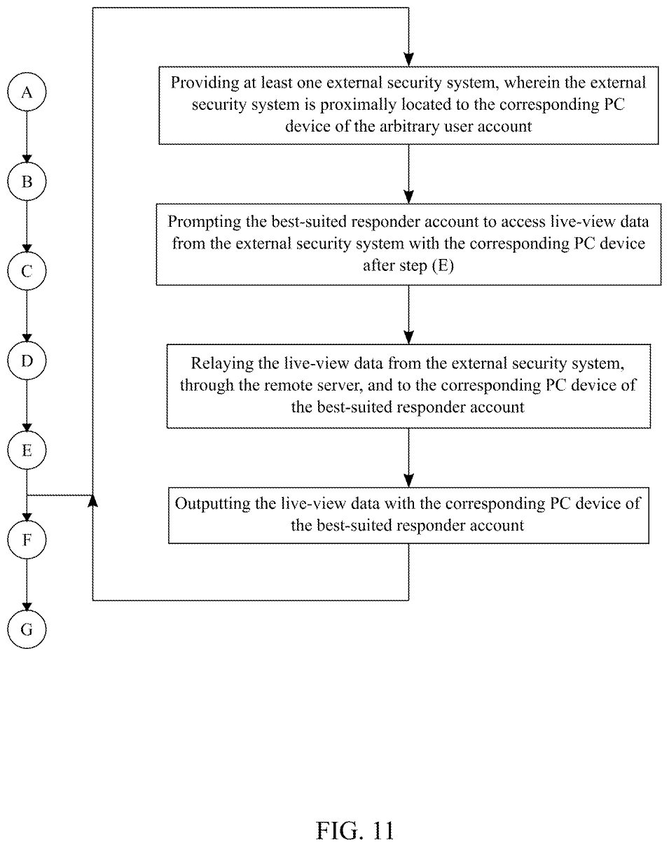

Live or real-time information is made even more beneficial by supplementing the content with proximal content captured on existing recording devices. To this end, at least one external security system is provided, wherein the external security system is proximally located to the corresponding PC device of the arbitrary user account, as represented in FIG. 11. The external security system may include any combination of a variety of cameras and recording devices capable of providing useful surveillance data. The best-suited responder account is prompted to access live-view data from the external security system with the corresponding PC device after Step E. Thus, the best-suited responder account can choose to control the flow of information from external security systems. The live-view data is relayed from the external security system, through the remote server, and to the corresponding PC device of the best-suited responder account. Thus, the remote server may not only store the data related to the emergency event, but also a variety of metadata, including, but not limited to, time of access, location of incident, location of access, and relevant IP address. Finally, the live-view data is outputted with the corresponding PC device of the best-suited responder account. Thus, the best-suited responder account can view the live-view data and react or prepare accordingly. This is particularly advantageous in situations such as medical emergencies where the best-suited responder needs to be able to communicate instructions in the best way possible.

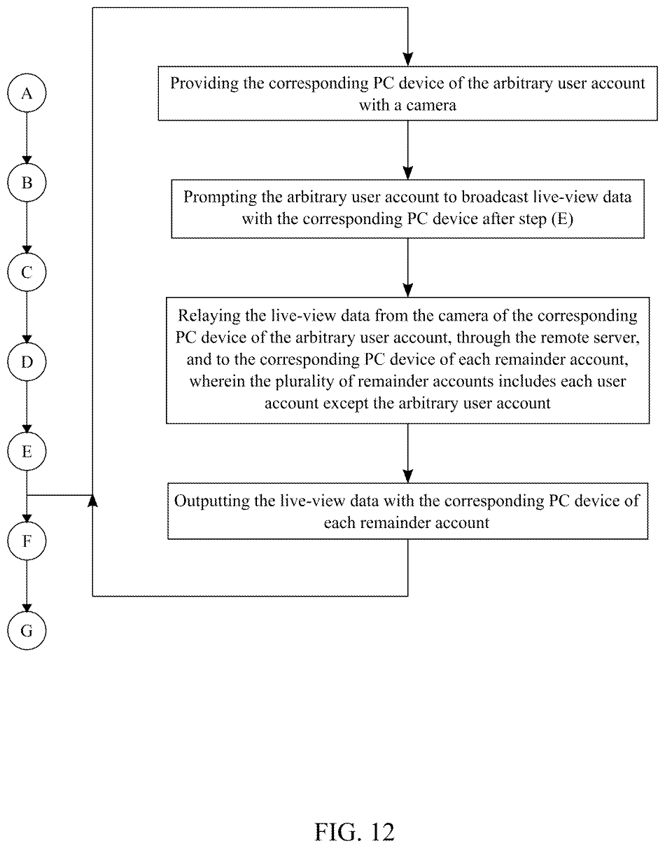

In some emergency situations, it is advantageous for the user to share captured video or content publicly. To achieve this, the corresponding PC device of the arbitrary user account is provided with a camera, as represented in FIG. 12. The arbitrary user account is prompted to broadcast live-view data with the corresponding PC device after Step E. Thus, the arbitrary user account may choose whether recorded content is meant to be sent out. The live-view data is relayed from the camera of the corresponding PC device of the arbitrary user account, through the remote server, and to the corresponding PC device of each remainder account, wherein the plurality of remainder accounts includes each user account except the arbitrary user account. Thus, the remote server provides each of the plurality of remainder accounts access to the live-view data within the emergency event. Finally, the live-view data is outputted with the corresponding PC device of each remainder account. In this way, the corresponding PC device of each remainder account is able to view the events being documented by the corresponding PC device of the arbitrary user account in real-time. Such an arrangement is particularly advantageous for a user who has been unlawfully apprehended by a police officer, or who is in a hostile environment.

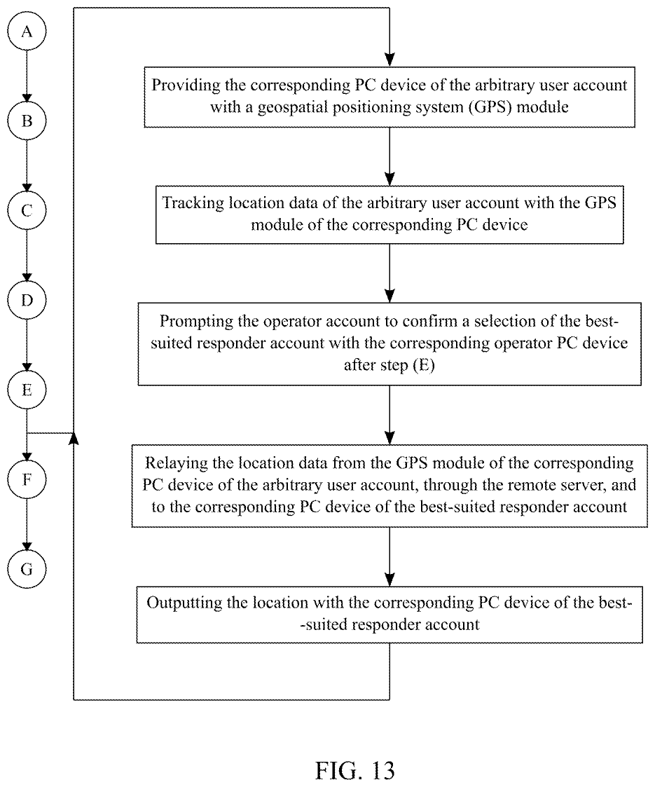

Among other potentially-useful information, an arbitrary user account may be especially likely to wish to communicate their location to first responders. To this end, the corresponding PC device of the arbitrary user account may be provided with a geospatial positioning system (GPS) module, as represented in FIG. 13. The GPS module denotes a device capable of relaying the precise geospatial coordinates of the user at a given time. Location data of the arbitrary user account is next tracked with the GPS module of the corresponding PC device. In this way, the GPS module is prepared to relay the user's location at a moment's notice. The best-suited responder account is prompted to access the location data with the corresponding PC device after Step E. This arrangement allows the best-suited responder to indicate their interest in approaching the location of the arbitrary user account. The location data is next relayed from the GPS module of the corresponding PC device of the arbitrary user account, through the remote server, and to the corresponding PC device of the best-suited responder account. This arrangement allows the best-suited responder account to access the location of the arbitrary user account as needed. Finally, the location data is outputted with the corresponding PC device of the best-suited responder account. Thus, the best-suited responder account may access and view the location of the arbitrary user account.

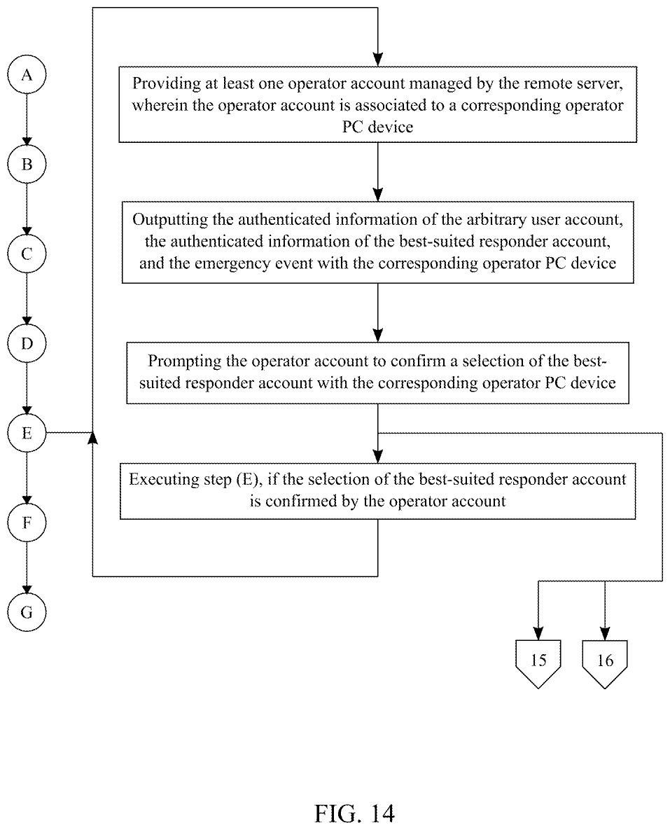

In many circumstances, it may be advantageous for a manual decision to be made regarding identifying the best-suited responder account during an emergency event entered by a personal account. To this end, at least one operator account managed by the remote server is provided, wherein the operator account is associated to a corresponding operator PC device, as represented in FIG. 14. The operator account relates to an individual capable of manually determining connections between the plurality of personal accounts and the plurality of responder accounts. The authenticated information of the arbitrary user account, the authenticated information of the best-suited responder account, and the emergency event are outputted with the corresponding operator PC device. Thus, the corresponding operator PC device controls the flow of connections between different user accounts. The operator account is prompted to confirm a selection of the best-suited responder account with the corresponding operator PC device. The operator account may use the information provided from the emergency event, as well as accompanying video where applicable, to make a decision as to which user account is the best-suited responder account. Finally, Step E is executed, if the selection of the best-suited responder account is confirmed by the operator account. Thus, the operator account determines the most appropriate connection for a given emergency event, particularly an emergency event activated from the arbitrary personal account.

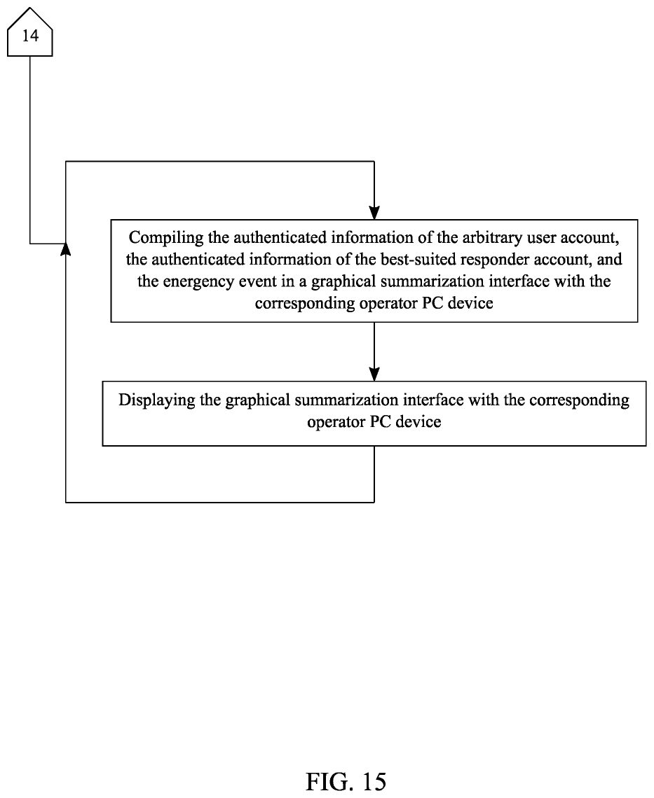

In order to facilitate efficient and effective communication between relevant parties, the present invention benefits from the ability to present information optimally to the operator account. To this end, the authenticated information of the arbitrary user account, the authenticated information of the best-suited responder account, and the emergency event are compiled in a graphical summarization interface with the corresponding operator PC device, as represented in FIG. 15. The graphical summarization interface may be any interface, such as a map overlaid with relevant data or information, interactive images, graphically displayed tabular data, or other such communication tools capable of fully capturing and optimally communicating necessary information. The graphical summarization interface is then displayed with the corresponding operator PC device. In this way, the graphical summarization interface is made accessible to the operator account.

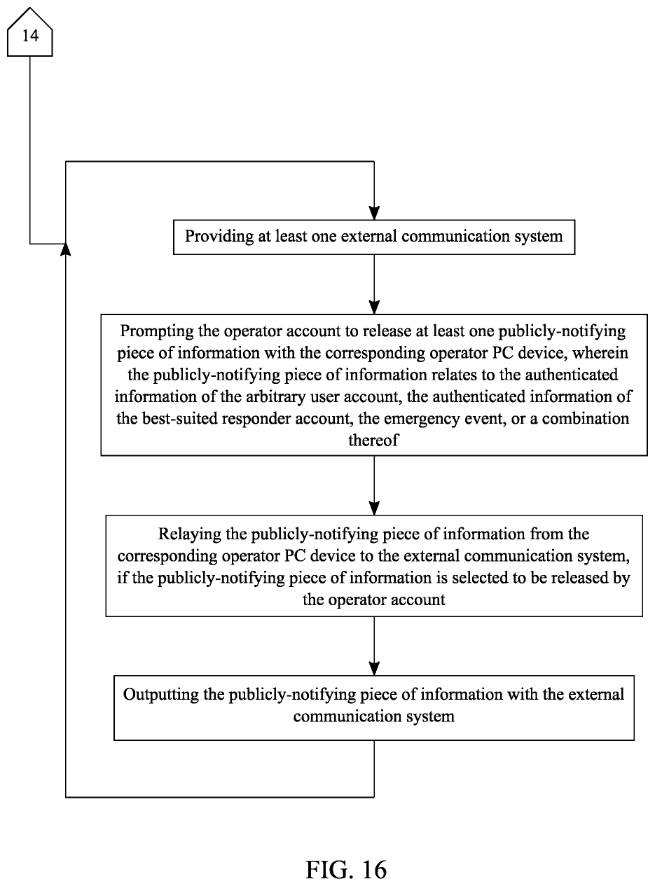

Furthermore, it may be in the best interest of the arbitrary user account to have the operator broadcast details of the emergency event publicly. To enable this, at least one external communication system is provided, as represented in FIG. 16. The at least one external communication system may relate to any of social media, radio, television stations, or other media. The operator account is next prompted to release at least one publicly-notifying piece of information with the corresponding operator PC device, wherein the publicly-notifying piece of information relates to the authenticated information of the arbitrary user account, the authenticated information of the best-suited responder account, the emergency event, or a combination thereof. In this way, relevant information gathered from all parties may be made available by the operator account. Subsequently, the publicly-notifying piece of information is relayed from the corresponding operator PC device to the external communication system, if the publicly-notifying piece of information is selected to be released by the operator account. This arrangement allows the publicly-notifying piece of information may be received by the external communication system. Finally, the publicly-notifying piece of information is outputted with the external communication system. Thus, the operator account may make the public aware of the publicly-notifying piece of information.

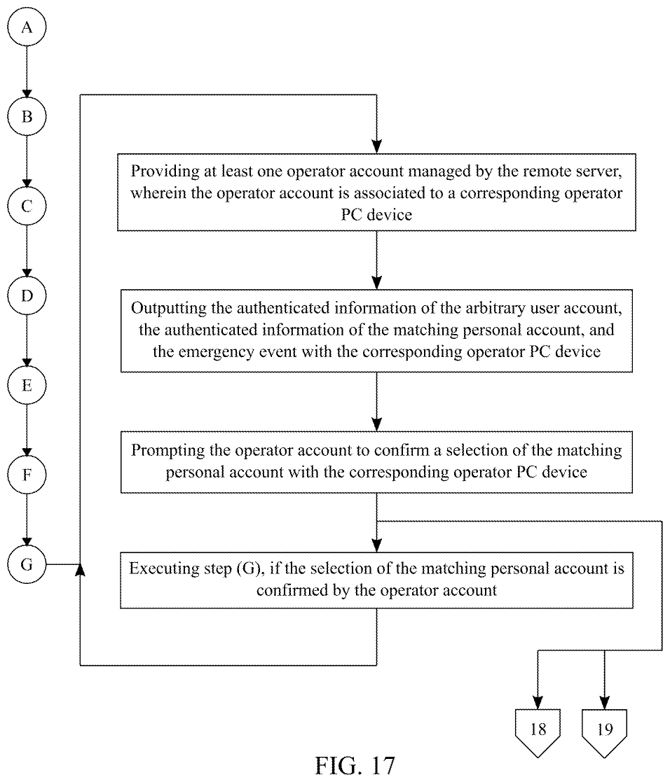

Alternatively, it may also be advantageous for manual decision-making regarding identifying a matching personal account during an emergency event entered by a responder account. To this end, at least one operator account managed by the remote server is provided, wherein the operator account is associated to a corresponding operator PC device, as represented in FIG. 17. The operator account relates to an individual capable of manually determining connections between the plurality of personal accounts and the plurality of responder accounts. The authenticated information of the arbitrary user account, the authenticated information of the best-suited responder account, and the emergency event are outputted with the corresponding operator PC device. Thus, the corresponding operator PC device manages the exchange of information between different user accounts. The operator account is prompted to confirm a selection of the matching personal account with the corresponding operator PC device. The operator account may use the information provided from the emergency event to make a decision, as well as accompanying video where applicable. Finally, Step G is executed, if the selection of the matching personal account is confirmed by the operator account. Thus, the operator account determines the most appropriate connection for a given emergency event, particularly an emergency event activated from an arbitrary responder account.

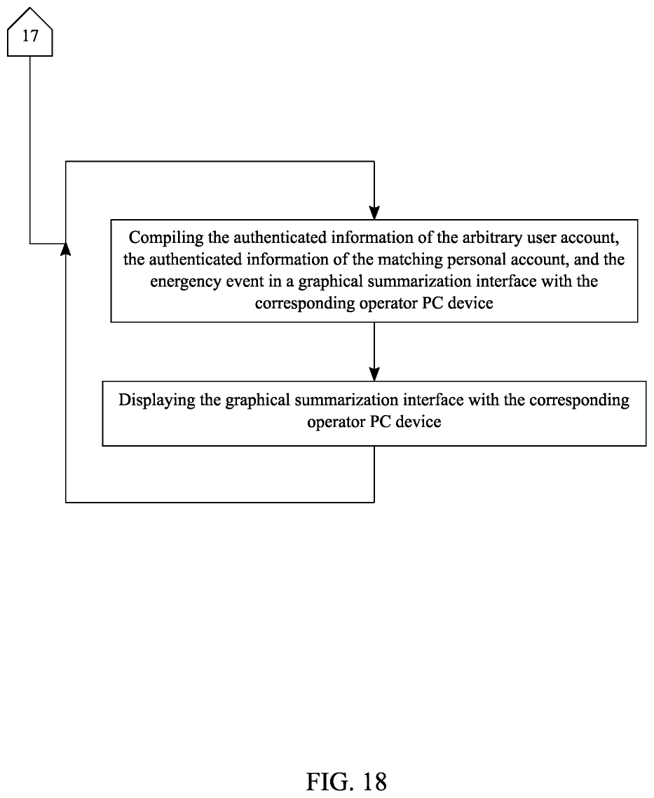

In order to facilitate efficient and effective communication between relevant parties, the present invention benefits from the ability to present information optimally to the operator account. To this end, the authenticated information of the arbitrary user account, the authenticated information of the matching personal account, and the emergency event are compiled in a graphical summarization interface with the corresponding operator PC device, as represented in FIG. 18. The graphical summarization interface may be any interface, such as a map overlaid with relevant data or information, interactive images, graphically displayed tabular data, or other such communication tools capable of fully capturing and optimally communicating necessary information. The graphical summarization interface is then displayed with the corresponding operator PC device. In this way, the graphical summarization interface is made accessible to the operator account.

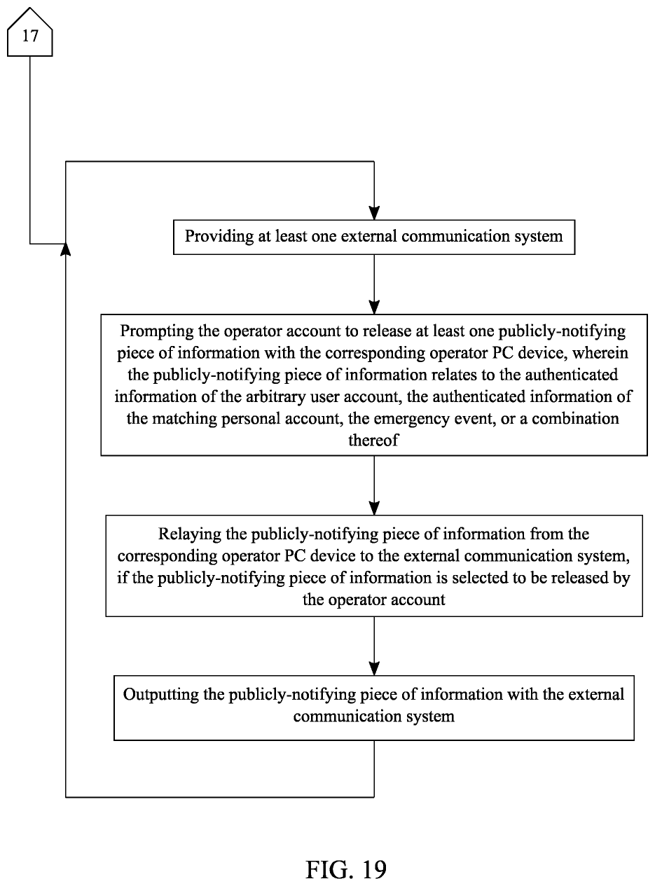

Furthermore, it may be in the best interest of the arbitrary user account to have the operator broadcast details of the emergency event publicly. To enable this, at least one external communication system is provided, as represented in FIG. 19. The at least one external communication system may relate to any of social media, radio, television stations, or other media. The operator account is next prompted to release at least one publicly-notifying piece of information with the corresponding operator PC device, wherein the publicly-notifying piece of information relates to the authenticated information of the arbitrary user account, the authenticated information of the matching personal account, the emergency event, or a combination thereof. In this way, relevant information gathered from all parties may be made available by the operator account. Subsequently, the publicly-notifying piece of information is relayed from the corresponding operator PC device to the external communication system, if the publicly-notifying piece of information is selected to be released by the operator account. This arrangement allows the publicly-notifying piece of information may be received by the external communication system. Finally, the publicly-notifying piece of information is outputted with the external communication system. Thus, the operator account may make the public aware of the publicly-notifying piece of information.

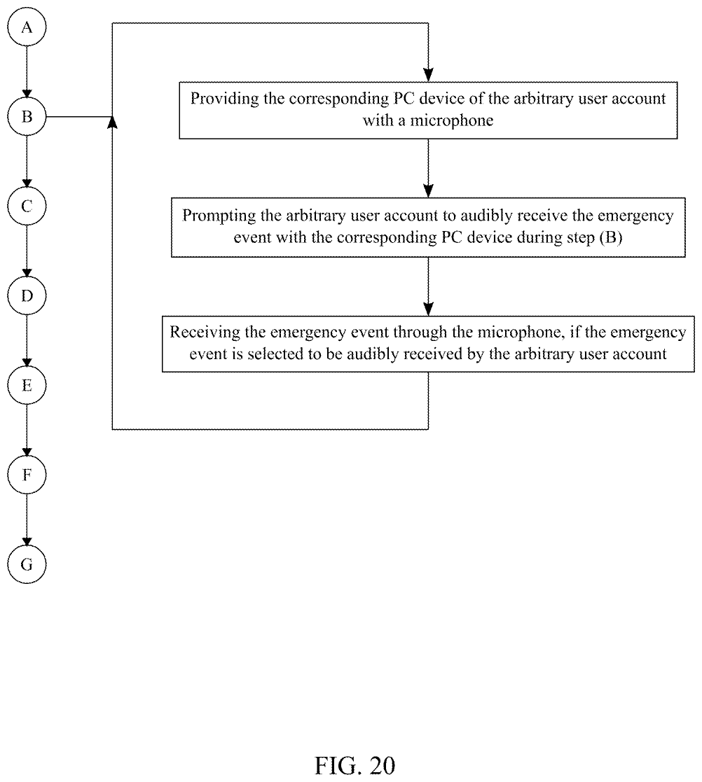

In a variety of scenarios and emergency situations, visual or video data may not be relevant, necessary, or available. To enable the provision of necessary assistance, the corresponding PC device of the arbitrary user account is provided with a microphone, as represented in FIG. 20. The microphone is a sensor capable of collecting and transmitting audio data. The arbitrary user account to audibly receive the emergency event with the corresponding PC device during Step B. In this way, the arbitrary user account consents to the exchange of information necessary in a given emergency situation. Finally, the emergency event is received through the microphone, if the emergency event is selected to be audibly received by the arbitrary user account. Thus, audio data relevant to an emergency situation may be collected and distributed to the best-suited responder account.

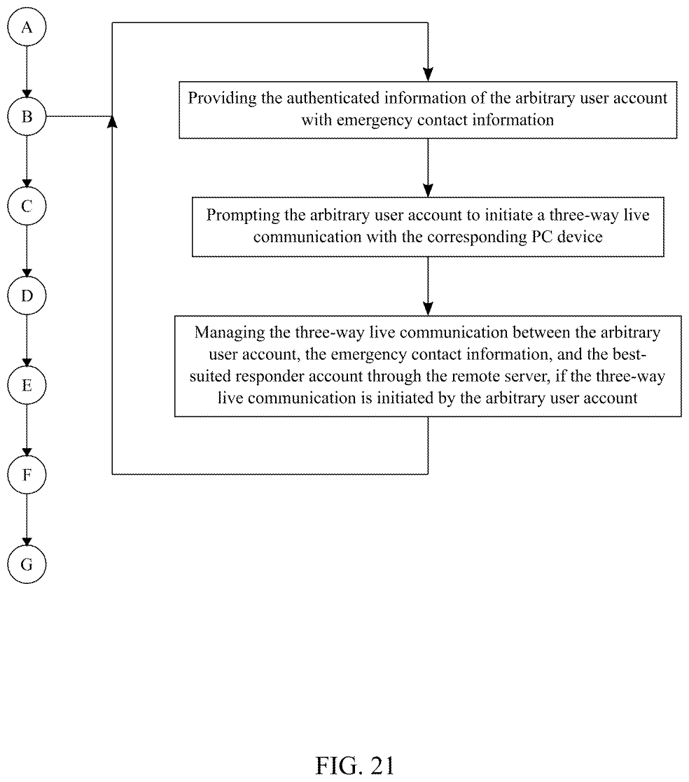

In many emergency situations, particularly in medical emergencies, timing is everything, and every second is considered precious. In order to facilitate treatment of medical emergencies and other emergencies that can be best managed by trained personnel, the authenticated information of the arbitrary user account is provided with emergency contact information, as represented in FIG. 21. In this way, an arbitrary user account may access someone else's emergency contacts as needed. The arbitrary user account is next prompted to initiate a three-way live communication with the corresponding PC device. In this way, the arbitrary user account can bring parties together to facilitate transmittance of information. Then, the three-way live communication is managed between the arbitrary user account, the emergency contact information, and the best-suited responder account through the remote server, if the three-way live communication is initiated by the arbitrary user account. In this arrangement, the arbitrary user account can be provided with information critical to assisting an individual in an emergency event, particularly a medical emergency.

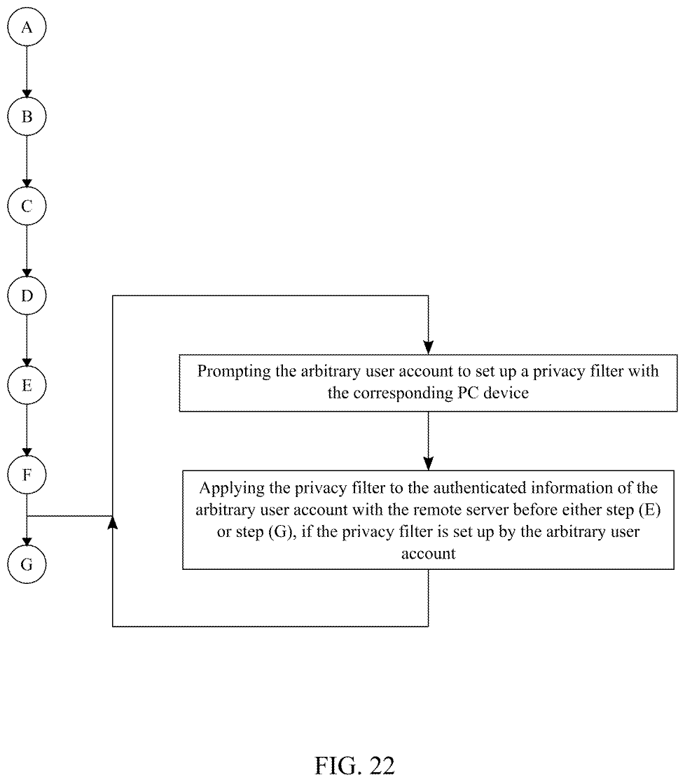

Oftentimes, individuals are concerned with the privacy of their information. To address this issue, the arbitrary user account is prompted to set up a privacy filter with the corresponding PC device, as represented in FIG. 22. The privacy filter may remove such entries as legal history, medical history, age, address, emergency contact information, and more, varying according to the different potential situations potentially corresponding to the emergency event. The privacy filter is applied to the authenticated information of the arbitrary user account with the remote server before either Step E or Step G, if the privacy filter is set up by the arbitrary user account. Thus, the user account can control the information that is accessible to others during transmittance of an emergency event, or as applicable.

Although the invention has been explained in relation to its preferred embodiment, it is to be understood that many other possible modifications and variations can be made without departing from the spirit and scope of the invention as hereinafter claimed.

What is claimed is:

1. A method for providing real-time communication with emergency services, the method comprises the steps of:
(A) providing a plurality of user accounts managed by at least one remote server, wherein each user account includes authenticated information and is associated with a corresponding personal computing (PC) device, and wherein the plurality of user accounts includes a plurality of personal accounts and a plurality of responder accounts;
(B) prompting each user account to enter an emergency event with the corresponding PC device;
(C) relaying the emergency event from the corresponding PC device of at least one arbitrary user account to the remote server, wherein the arbitrary user account is any account from the plurality of user accounts;
(D) comparing the emergency event to the authenticated information of each responder account with the remote server in order to identify at least one best-suited responder account from the plurality of responder accounts, if the arbitrary user account is from the plurality of personal accounts;
(E) outputting the emergency event and the authenticated information of the arbitrary user account with the corresponding PC device of the best-suited responder account;
(F) comparing the emergency event to the authenticated information of each personal account with the remote server in order to identify at least one matching personal account from the plurality of personal accounts, if the arbitrary user account is from the plurality of responder accounts; and
(G) outputting the emergency event and the authenticated information of the arbitrary user account with the corresponding PC device of the matching personal account.

2. The method for providing real-time communication with emergency services, the method as claimed in claim 1 comprises the steps of:
providing the emergency event as a description of a theft;
identifying the best-suited responder account with the remote server during step (D), if the authenticated information of the best-suited responder account includes a set of law enforcement credentials; and
further outputting a dispatch notification with the corresponding PC device of the best-suited responder account during step (E).

3. The method for providing real-time communication with emergency services, the method as claimed in claim 1 comprises the steps of:
providing the emergency event as a description of a medical situation;
identifying the best-suited responder account with the remote server during step (D), if the authenticated information of the best-suited responder account includes a set of medical credentials; and
further outputting a dispatch notification with the corresponding PC device of the best-suited responder account during step (E).

4. The method for providing real-time communication with emergency services, the method as claimed in claim 1 comprises the steps of:
providing the emergency event as a description of a law enforcement situation;
further prompting the arbitrary user account to enter personal identifying information with the corresponding PC device, if the arbitrary user account is from the plurality of responder accounts;
relaying the personal identifying information from the corresponding PC device of the arbitrary user account to the remote server, if the personal identifying information is entered by the arbitrary user account; and
identifying the matching personal account with the remote server during step (F), if the authenticated information of the matching personal account includes the personal identifying information.

5. The method for providing real-time communication with emergency services, the method as claimed in claim 4 comprises the step of:
outputting the authenticated information of the arbitrary user account with the corresponding PC device of the matching personal account.

6. The method for providing real-time communication with emergency services, the method as claimed in claim 1 comprises the step of:
outputting the authenticated information of the arbitrary user account with the corresponding PC device of the matching personal account.

7. The method for providing real-time communication with emergency services, the method as claimed in claim 1 comprises the steps of:
providing the corresponding PC device of the arbitrary user account with a camera;
capturing emergency visual data with the camera before step (B); and
appending the emergency visual data into the emergency event with the corresponding PC device of the arbitrary user account.

8. The method for providing real-time communication with emergency services, the method as claimed in claim 1 comprises the steps of:
providing the corresponding PC device of the arbitrary user account with a camera;
prompting the best-suited responder account to access live-view data for the arbitrary user account with the corresponding PC device after step (E);
relaying the live-view data from the camera of the corresponding PC device of the arbitrary user account, through the remote server, and to the corresponding PC device of the best-suited responder account; and
outputting the live-view data with the corresponding PC device of the best-suited responder account.

9. The method for providing real-time communication with emergency services, the method as claimed in claim 1 comprises the steps of:
providing at least one external security system, wherein the external security system is proximally located to the corresponding PC device of the arbitrary user account;
prompting the best-suited responder account to access live-view data from the external security system with the corresponding PC device after step (E);
relaying the live-view data from the external security system, through the remote server, and to the corresponding PC device of the best-suited responder account; and
outputting the live-view data with the corresponding PC device of the best-suited responder account.

10. The method for providing real-time communication with emergency services, the method as claimed in claim 1 comprises the steps of:
providing the corresponding PC device of the arbitrary user account with a camera;
prompting the arbitrary user account to broadcast live-view data with the corresponding PC device after step (E);
relaying the live-view data from the camera of the corresponding PC device of the arbitrary user account, through the remote server, and to the corresponding PC device of each remainder account, wherein the plurality of remainder accounts includes each user account except the arbitrary user account; and
outputting the live-view data with the corresponding PC device of each remainder account.

11. The method for providing real-time communication with emergency services, the method as claimed in claim 1 comprises the steps of:
providing the corresponding PC device of the arbitrary user account with a geospatial positioning system (GPS) module;
tracking location data of the arbitrary user account with the GPS module of the corresponding PC device;
prompting the best-suited responder account to access the location data with the corresponding PC device after step (E);
relaying the location data from the GPS module of the corresponding PC device of the arbitrary user account, through the remote server, and to the corresponding PC device of the best-suited responder account; and
outputting the location with the corresponding PC device of the best-suited responder account.

12. The method for providing real-time communication with emergency services, the method as claimed in claim 1 comprises the steps of:
providing at least one operator account managed by the remote server, wherein the operator account is associated to a corresponding operator PC device;
outputting the authenticated information of the arbitrary user account, the authenticated information of the best-suited responder account, and the emergency event with the corresponding operator PC device;
prompting the operator account to confirm a selection of the best-suited responder account with the corresponding operator PC device; and
executing step (E), if the selection of the best-suited responder account is confirmed by the operator account.

13. The method for providing real-time communication with emergency services, the method as claimed in claim 12 comprises the steps of:
compiling the authenticated information of the arbitrary user account, the authenticated information of the best-suited responder account, and the emergency event in a graphical summarization interface with the corresponding operator PC device; and
displaying the graphical summarization interface with the corresponding operator PC device.

14. The method for providing real-time communication with emergency services, the method as claimed in claim 12 comprises the steps of:
providing at least one external communication system;
prompting the operator account to release at least one publicly-notifying piece of information with the corresponding operator PC device, wherein the publicly-notifying piece of information relates to the authenticated information of the arbitrary user account, the authenticated information of the best-suited responder account, the emergency event, or a combination thereof;
relaying the publicly-notifying piece of information from the corresponding operator PC device to the external communication system, if the publicly-notifying piece of information is selected to be released by the operator account; and
outputting the publicly-notifying piece of information with the external communication system.

15. The method for providing real-time communication with emergency services, the method as claimed in claim 1 comprises the steps of:
providing at least one operator account managed by the remote server, wherein the operator account is associated to a corresponding operator PC device;
outputting the authenticated information of the arbitrary user account, the authenticated information of the matching personal account, and the emergency event with the corresponding operator PC device;
prompting the operator account to confirm a selection of the matching personal account with the corresponding operator PC device; and
executing step (G), if the selection of the matching personal account is confirmed by the operator account.

16. The method for providing real-time communication with emergency services, the method as claimed in claim 15 comprises the steps of:
compiling the authenticated information of the arbitrary user account, the authenticated information of the matching personal account, and the emergency event in a graphical summarization interface with the corresponding operator PC device; and
displaying the graphical summarization interface with the corresponding operator PC device.

17. The method for providing real-time communication with emergency services, the method as claimed in claim 15 comprises the steps of:
providing at least one external communication system;
prompting the operator account to release at least one publicly-notifying piece of information with the corresponding operator PC device, wherein the publicly-notifying piece of information relates to the authenticated information of the arbitrary user account, the authenticated information of the matching personal account, the emergency event, or a combination thereof;
relaying the publicly-notifying piece of information from the corresponding operator PC device to the external communication system, if the publicly-notifying piece of information is selected to be released by the operator account; and
outputting the publicly-notifying piece of information with the external communication system.

18. The method for providing real-time communication with emergency services, the method as claimed in claim 1 comprises the steps of:
providing the corresponding PC device of the arbitrary user account with a microphone;
prompting the arbitrary user account to audibly receive the emergency event with the corresponding PC device during step (B); and
receiving the emergency event through the microphone, if the emergency event is selected to be audibly received by the arbitrary user account.

19. The method for providing real-time communication with emergency services, the method as claimed in claim 1 comprises the steps of:
providing the authenticated information of the arbitrary user account with emergency contact information;
prompting the arbitrary user account to initiate a three-way live communication with the corresponding PC device; and
managing the three-way live communication between the arbitrary user account, the emergency contact information, and the best-suited responder account through the remote server, if the three-way live communication is initiated by the arbitrary user account.

20. The method for providing real-time communication with emergency services, the method as claimed in claim 1 comprises the steps of:
prompting the arbitrary user account to set up a privacy filter with the corresponding PC device; and
applying the privacy filter to the authenticated information of the arbitrary user account with the remote server before either step (E) or step (G), if the privacy filter is set up by the arbitrary user account.
